(19) United States
(12) Patent Application Publication
(10) Pub. No.: US 2005/0065582 A1
(43) Pub. Date: Mar. 24, 2005

(54) Apparatus and method for providing a cooling effect to the skin

(76) Inventor: John Thomas Blackburn, Clearwater, FL (US)

Correspondence Address: DENNIS G. LAPOINTE;LAPOINTE LAW GROUP, PL, PO BOX 1294, TARPON SPRINGS, FL, 34688-1294, US

(21) Appl. No.: 10/670,491

(22) Filed: Sep. 24, 2003

(51) Int. Cl. A61F 7/00

(52) U.S. Cl. 607/104

(57) ABSTRACT

The invention is a method, inclusive of its associated device or apparatus, of more effectively cooling the human body by the process of an enhanced evaporation process. The method provides for the spraying a generally wide pattern of a fine mist spray of de-mineralized water on the skin to be cooled from either a non-pressurized container having a manual pump spray for discharging the water from the container, or a pressurized container having a propellant mixed with the de-mineralized water, where the pressurized container further has a manually operated actuator for discharging the water from the container. In the latter case, the propellant is one of nitrogen, carbon dioxide and air, preferably nitrogen.

This invention relates to an apparatus and method for providing a refreshing cooling effect to a user's skin by spraying a fine mist of de-mineralized water onto the skin.

### SUMMARY OF THE INVENTION

Generally, the invention includes a product or apparatus, which is a self-contained can or other suitable container of de-mineralized water, where the water can be applied by use of a manually operated pump without a propellant or the water can be pressurized by a propellant such as dry nitrogen, carbon dioxide gas or air and applied under pressure from the propellant. A manually operated pump is known and typically includes an integral valve. As herein understood, such pumps include those spray devices that incorporate a bias handle which is squeezed as well as the type of pumps sometimes used on bottles such as window cleaner bottles.

Although not harmful if ingested, the present invention is intended for topical use only. When sprayed, the water is atomized out of the container and directed onto the skin in a wide, fine mist for an instant and lasting cooling affect.

### DETAILED DESCRIPTION OF THE INVENTION

Although any type of water may be used, the object of the invention was to provide a pure form of water to eliminate the potential for skin damage caused by hard water or water containing high mineral contents.

Accordingly, the preferred embodiment includes the use of the purest form of water and a propellant that allowed for the highest fill point in the container. De-mineralized water significantly reduces the hardness of the water by eliminating the minerals and essentially reducing the water hardness to zero. De-mineralized water is also commonly known in industry as “pure water” and is often used to clean objects in “clean rooms” when high quality cleanliness is required or in laboratory work. It has never been used to cool down the skin in the form of a spray mist.

The preferred embodiment also includes a pump actuator or valve and actuator for the pressurized containers, both styles together with a nozzle orifice that allows for the spraying of the water on the skin in a wide, fine mist pattern. Generally, people understand the difference between a concentrated spray, fine mist spray and such a spray that covers a wide area by just relating to various adjustments that can be made to hose nozzle and to other products as found in the cosmetic industry such as hair sprays, etc. Therefore, one skilled in the art can readily understand what is meant by a wide, fine mist spray when reading this application, without the need for further elaboration.

Typically, de-mineralized water is process through a filtering and de-ionization process using charcoal filters, inline micron filters and resin beds for the de-ionization process at ambient temperatures. The charcoal filters remove the organic material and the de-ionization process and inline micron filters strip the water of all contaminants, including dissolved minerals.

Using a de-mineralized supply of water, the processed water is filled into the container. If a pressurized container is used such as a can, the can is sealed with the valve and actuator and it is charged with the propellant such as dry nitrogen to a pressure of about 90 to 100 pounds per square inch.

Although other formulations can be used, an effective formulation is achieved by weight at a ratio of about 99.95% water and 0.05% dry nitrogen. However, the nitrogen is not weighed into the unit but rather, it is time released until it achieves the desired pressure.

The cooling effect achieved is due to the water itself. Since the de-mineralized water is not laden with minerals, it has an affinity to the sweat and minerals that the human body produces and evaporates at a lower temperature from the skin surface, thus producing an earlier or faster cooling effect. This effect, coupled with the fine mist produced through the configuration of the valve and actuator (or pump) and outlet orifice provides large surface area coverage, which further allows the water or enhances the ability of the water to evaporate more quickly.

Similar products exist, however, those products utilize much poorer quality waters that are not pure and contain several hard minerals with measurements of Ca²⁺ and Mg²⁺. Water containing such impurities cannot evaporate as quickly because it is literally weighted down by them. The body absorbs hard minerals that it needs internally and there is no advantage to applying them onto the skin. The human body naturally cools itself by process of evaporation. The present invention enables or facilitates that process more rapidly. In doing so, less fluid is lost from sweating thus slowing down the process of dehydration or heat stroke or avoiding it all together.

When different quality waters were tested, the de-mineralized water not only felt cooler when sprayed onto the skin, but also evaporated more quickly. This study was conducted with tap water, spring water and de-mineralized water in identical trigger type spray bottles at identical temperatures. The samples were labeled A, B and C.
A=De-mineralized water
B=Spring water
C=Tap water

Each sample bottle was set to spray the same width mist and was held six inches away from the forearm and sprayed one time onto the skin. The people participating in the study did not know the purity or type of the water that they were spraying at that time and could choose their sample in any order. Four people were chosen to participate and the samples were graded in two ways.
a. How cool the water felt when sprayed onto the skin; and
b. How long it took the water to evaporate.

Three of the four participants remarked that sample A felt cooler when it was sprayed on the skin. In addition, sample A, the de-mineralized water evaporated completely in one minute and six seconds on average, about twenty-four seconds faster than spring and tap water.

In addition to using this product at room temperature it can be stored in the refrigerator or in a cooler for an intensified cooling effect. It is not refillable and it is environmentally safe, especially when using nitrogen as the propellant.

Additional uses for this product can include, but are not limited to, the homogeneous infusion of fragrances being introduced into the product, or a sun blocking factor material or a skin care material such as an aloe vera liquid and possibly even insect repellant.

Accordingly, the invention is a method, inclusive of its associated device or apparatus, of more effectively cooling the human body by the process of an enhanced evaporation process. The method provides for the spraying a generally wide pattern of a fine mist spray of de-mineralized water on the skin to be cooled from either a non-pressurized container having means for manually pumping and discharging the water from the container, or a pressurized container having a propellant mixed with the de-mineralized water, where the pressurized container further has actuator means for manually activating the discharge of the water from the container. In the latter case, the propellant is one of dry nitrogen and carbon dioxide, preferably dry nitrogen.

Manual spray pumps for spray bottle are known in the art and are typical of what is found in grocery or hardware stores. The nozzles are generally adjustable to obtain a relatively narrow concentrated spray pattern or a generally wide, misty type of spray pattern. Actuators for pressurized containers are also known in the art such as those found in paint spray cans. Typically it is a valve in the upper neck of the can which is activated by pushing the portion of the spray mechanism in a downward direction to open the valve and allow the propellant to escape thereby discharging the content of the container, in this case, the de-mineralized water. The orifice and valve are designed to allow for a wide, fine mist spray in lieu of a more concentrated spray.

1. A method of more effectively cooling the human body by the process of an enhanced evaporation process, the method comprising:
spraying a generally wide pattern of a fine mist spray of de-mineralized water on the skin to be cooled from one of:
a non-pressurized container having means for manually pumping and discharging the water from the container; and
a pressurized container having a propellant mixed with the de-mineralized water, the pressurized container further having means for manually activating the discharge of the water from the container.

2. The method according to claim 1, wherein the propellant is nitrogen.

3. The method according to claim 1, wherein the propellant is selected from the group comprising carbon dioxide and air.

4. The method according to claim 1, wherein the weighted ratio of de-mineralized water to the propellant in the container is approximately 99.95% de-mineralized water and 0.05% propellant.

5. The method according to claim 1, wherein when a propellant is used, the container is pressurized to a pressure of about 90 to 100 psi.

6. The method according to claim 1, wherein the de-mineralized water further includes liquid material selected from the group consisting of fragrances, sun blocking factor materials, skin care materials and insect repellant materials.

7. An apparatus for more effectively cooling the human body by the process of an enhanced evaporation process, the apparatus comprising:
a container containing de-mineralized water;
the container having means for spraying a generally wide pattern of a fine mist spray of said de-mineralized water on the skin to be cooled;
the container being one of:
a non-pressurized container having means for manually pumping and discharging its liquid content from the container; and
a pressurized container having a propellant mixed with the de-mineralized water, the pressurized container further having means for manually activating the discharge of the water from the container.

8. The apparatus according to claim 7, wherein the propellant is nitrogen.

9. The apparatus according to claim 7, wherein the propellant is selected from the group comprising carbon dioxide and air.

10. The apparatus according to claim 7, wherein the weighted ratio of de-mineralized water to the propellant in the container is approximately 99.95% de-mineralized water and 0.05% propellant.

11. The apparatus according to claim 7, wherein when a propellant is used, the container is pressurized to a pressure of about 90 to 100 psi.

12. The apparatus according to claim 7, wherein the de-mineralized water further includes liquid material selected from the group consisting of fragrances, sun blocking factor materials, skin care materials and insect repellant materials.
